(19) United States
(12) Patent Application Publication
(10) Pub. No.: US 2011/0151552 A1
(43) Pub. Date: Jun. 23, 2011

(54) BIOREACTORS

(75) Inventors: TIEYING JIANG, Shrewsbury, MA (US); JEFFREY CARTER, Hopkinton, MA (US); SHUJIAN YI, Nashua, NH (US)

(73) Assignee: GE HEALTHCARE BIO-SCIENCES CORP., PISCATAWAY, NJ (US)

(21) Appl. No.: 12/645,534

(22) Filed: Dec. 23, 2009

(51) Int. Cl. C12M 1/00

(52) U.S. Cl. 435/289.1

(57) ABSTRACT

The present invention relates to improvements of bioreactor bags for cell cultivation. The invention provides an inflatable bioreactor bag for cell cultivation comprised of a top and a bottom sheet of polymer material that are joined along their edges to form a sealed bag, wherein two opposing edges are formed as clamping edges to allow clamping of the bioreactor bag to a rocker type bioreactor, wherein the top sheet is at least 5% longer than the bottom sheet between the clamping edges. The bioreactor bags provided by the invention avoid formation of undesired wrinkles or creases which otherwise may lead to fatigue of the plastic and eventually fracture.

### TECHNICAL FIELD

The present invention relates to improved single-use bioreactors comprising disposable plastic bags for cell cultivation. The bioreactor bags provided by the invention avoid formation of undesired wrinkles or creases which otherwise may lead to fatigue of the plastic and eventually fracture.

### BACKGROUND OF THE INVENTION

The bioprocessing industry has traditionally used stainless steel systems and piping in manufacturing processes for fermentation and cell culture. These devices are designed to be steam sterilized and reused. Cleaning and sterilization are however costly labor-intensive operations. Moreover, the installed cost of these traditional systems with the requisite piping and utilities is often prohibitive. Furthermore, these systems are typically designed for a specific process, and cannot be easily reconfigured for new applications. These limitations have led to adoption of a new approach over the last ten years—that of using plastic, single-use disposable bags and tubing, to replace the usual stainless steel tanks.

In particular bioreactors, traditionally made of stainless steel, have been replaced in many applications by disposable bags which are rocked to provide the necessary aeration and mixing necessary for cell culture. These single-use bags are typically sterile and eliminate the costly and time-consuming steps of cleaning and sterilization. The bags are designed to maintain a sterile environment during operation thereby minimizing the risk of contamination.

Bags containing sterile fluids are used in the bioprocessing industry for formulation, storage, transfer, processing, and transportation. Sterile conditions must be maintained during these operations, and the bags are usually sealed to prevent contamination. Commonly used bags are of the “pillow style,” mainly because these can be manufactured at low cost by seaming together two flexible films of plastic.

Current disposable cell culture chambers (bags) are designs of 2-D structures. When bags are inflated with air and medium as well as fixed onto bioreactors, creases around corner area are formed and these corner creases move back and forth with the rocking motion of bioreactor. Some creases will develop fatigue crazes after more than several thousands of cyclic motions. Media leakage and contamination will eventually occur once fatigue crazes penetrate deeply through every constituent layer of the polymer film of the bag.

US2009/0188211A1 (Xcellerex Inc) describes systems and methods for containing and manipulating fluids, such as those involving collapsible bags and rigid containers. Bag wrinkle removing systems are described comprising pneumatically operable bladders that may modify or change the shape of the collapsible bag in order to prevent formation of folds and wrinkles therein.

### SUMMARY OF THE INVENTION

The present inventors have found that formation of corner creases is due to high corner stress of inflated bags. The present invention provides disposable cell culture bags that will prevent or minimize the formation of these creases by addressing the corner stress issue. The strategy is to divert corner stress to other areas of the bag or to reinforce corner area so that the rim would not fold itself. Extra structures on the corners and/or side rims of the bag have been found to form a very smooth contour on the corners (minimal to no creases). As a result of significant reduction or removal of corner creases, these new bags will not have any fatigue failure (cracking, delaminating, leaking) during cell culture process.

Thus, in a first aspect the present invention relates to an inflatable bioreactor bag for cell cultivation comprised of a top and a bottom sheet of polymer material that are joined along their edges to form a sealed bag, wherein two opposing edges are formed as clamping edges to allow clamping of said bioreactor bag to a rocker type bioreactor, wherein the top sheet is at least 5% longer than the bottom sheet between the clamping edges. In all the embodiments of the invention the bottom part is substantially flat and the upper part is enlarged compared to the bottom part.

In one embodiment, the bioreactor bag may comprise intermediate side sheets interconnecting the top and a bottom sheet, wherein the top sheet is at least 5% longer than the bottom sheet between the clamping edges.

The top and/or bottom sheet may be integrated with the side sheets.

In a further embodiment, the side sheet has one edge of equal length as the bottom sheet.

In all of these embodiments, the length between the non-clamping edges of the top sheet are formed to be of equal length as the bottom sheet between the non-clamping edges.

### BRIEF DESCRIPTION OF THE DRAWINGS

FIG. 1 is a schematic view of an embodiment of a disposable bag according to the invention with enlarged top sheet.

FIG. 2 is a cross-sectional view of the bag in FIG. 1 seen from the non-clamping or non-bar edge wherein the side sheet has a curved edge.

FIG. 3 is a cross-sectional view from the non-clamping edge of a further embodiment of a disposable bag wherein the side sheet is triangular.

FIG. 4 is a cross-sectional view from the non-clamping edge of a further embodiment of a disposable bag wherein the side sheet is rectangular.

FIG. 5 is a cross-sectional view from the non-clamping edge of a further embodiment of a disposable bag wherein the side sheet is pentagon.

FIG. 6 is a cross-sectional view from the non-clamping edge of a further embodiment of a disposable bag wherein the side sheet is trapezoid.

### DETAILED DESCRIPTION OF THE INVENTION

The present invention is an improvement of a bioreactor bag that consists of a pre-sterilized flexible plastic bag in which cells are cultivated. The bag is partially filled with growth media and the remainder of the bag is continuously purged with air or other oxygen-rich gas. The bag is placed on a platform that can be rocked to and fro. The rocking motion promotes wave formation in the bag, which provides liquid mixing and enhances oxygen transfer from the headspace gas to the liquid phase where it is essential for cell growth and metabolism. The air in the bag performs several functions: 1) allows the formation of surface waves promoting oxygen transfer; 2) continually provides fresh oxygen into the bag and sweeps out gaseous metabolic products and 3) inflates the bag to a rigid form which reduces foam formation and promotes liquid mixing.

By using a disposable bag as the only contact surface for the cells, the bioreactor bag provides excellent containment and eliminates labor intensive cleaning and sterilization. Lack of any mechanical parts except for the rocking platform dramatically reduces cost and maintenance. The gentle wave agitation provides an intrinsically low shear environment. Aeration is also performed without generating cell-damaging bubbles.

The invention is useful for animal, plant, microbial, and insect cell culture, both in free suspension as well for anchorage-dependent systems. It is very suitable for virus and pathogen cultivation because of the high degree of containment.

The bioreactor consists of a disposable pre-sterilized plastic bag that rests on rocking platform. The platform may be made of stainless steel or consist of any other rigid material such as, plastic, fiberglass, aluminum, etc.

Restraining straps prevent the bag from slipping off the platform. The inlet air pressure and outlet air pressure control will prevent over/under inflation. Other means to secure the bag such as a rigid holder, tape, or sleeve may also be used. It is critical that the bag be prevented from over inflation otherwise the bottom surface will not conform to the flat profile of the platform and poor wave action will result. It is likewise important to avoid under inflation, as an under inflated bag will have many wrinkles and will flex excessively, both of which lead to premature failure. For proper wave motion, it is critical that the bag not be completely full of liquid. In the present embodiments the liquid phase may comprise 10 to 80% of the total bag volume.

The platform may contain an integral heater controlled by a temperature sensor and controller that can be used to maintain a predetermined temperature in the cultivation chamber. The rocking action ensures that a uniform temperature is achieved in the culture fluid. Humidity of the inlet gas may be controlled to reduce evaporation. Other gases, such as carbon dioxide, may be introduced into the chamber to control pH and other environmental conditions.

Cultivation is done by inflating the bag with air, then introducing liquid media into the bag. The culture is then introduced into the bag. Rocking rate and aeration are then set at predetermined values. Samples may be withdrawn by connecting a syringe to a sampling port. Virus inoculums or media additions can also be added through this port at appropriate times during the cultivation. Harvesting is done by pumping out the cell culture broth. The next batch can be initiated immediately by placing a new bag on the platform.

When bags are fixed on bioreactors and inflated with air, stress at corner is normally higher than that in area further away from corners. Two opposite bag rims are usually supported with bars and these supported bag rims ore edges are attached to the rocking plate for example by clamping down or by other means. The problem is that high corner stresses cause folding of non-bar-supported bag rims and create creases on corners. Fatigue cracking on 50 L, 200 L, and 1000 L WAVE CELLBAG™ bioreactor bags was reported in a few days to greater than 20 days of cell culture process. This may lead to scrapping of the bioreactor contents and economic loss for the user of the bioreactor. The present invention proves new solutions to minimize the formation of corner creases to significantly enhance fatigue resistance of bags.

The inventors found that formation of corner creases is also partly due to larger bag dimensions of 50 L, 200 L, and 1000 L bags than the corresponding bag holders. A quick and easy solution is to enlarge bag holders so that bags are stretched tightly and no freedom is left for bag to fold its rims. However, this approach will alter medium flow pattern and mass transfer between air and medium inside the bag, which is not desired.

The solution to the problem according to the invention will minimize or not alter medium flow and mass transfer inside bag by alleviating corner stress or to strengthening rim of current bags by changing bag design; no bag mounting technique change is needed. In the drawings below are six examples of these new designs.

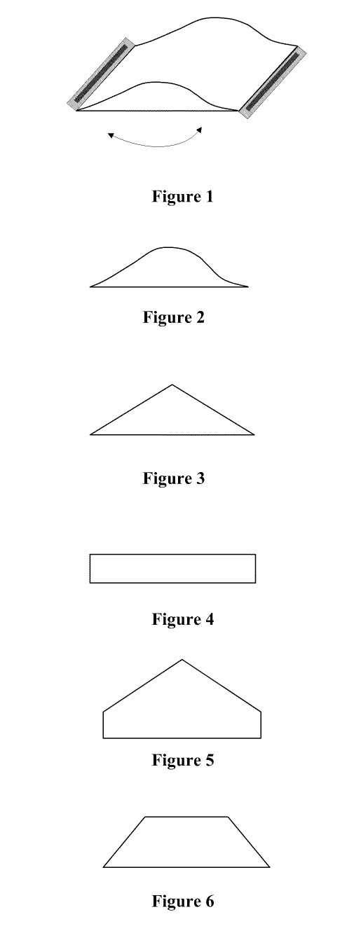

FIG. 1 schematically shows a disposable bag according to the invention comprising a bottom flat sheet and a top sheet. The top sheet is enlarged on two sides compared to the bottom sheet. In these two sides, the top sheet is attached to side sheets and side sheets attached to bottom sheet to form a 3-D structure. The other two opposing edges are formed by sealing top sheet and bottom sheet together, shown in the FIG. 1 as grey with a black insertion, referred to as the clamping edges, which are meant to be clamped down on rocking device to secure the bag on the device. In FIG. 1, the black insertion is a rigid polymer rod to hold the bag in position while the bioreactor is rocking. The strength of rigid polymer rods is skilfully tuned such that the polymer rods would provide sufficient rigidity during cell culture application. These polymers must also be gamma stable. Polymers for reinforcement rod are thermoplastic or thermosetting materials, such as acrylic, nylon, polyethylene, and polyvinyl chloride (PVC), etc. The bioreactor bag may be produced of transparent flexible films, such as single or multi-layered low density polyethylene (LDPE), linear low density polyethylene (LLDPE), ultra low density polyethylene (ULDPE), poly(ethylene-vinyl alcohol) (EVOH), polyvinylidene dichloride (PVDC), poly(ethylene-vinyl acetate) (EVA), nylon, and polyethylene terephthalate (PET). Some of these polymers might be USP class VI certified.

As appears from the cross-sectional view in FIG. 2 the bottom sheet is flat compared to the top sheet. The top sheet is at least 5% longer between the clamping edges than the bottom sheet so that it can attach to the curved edge of the side sheet.

FIGS. 3-6 show cross sectional views and side sheet geometry of various alternative embodiments of a bioreactor bag according to the invention. In FIG. 3 the side sheet is triangular. In FIG. 4, the side sheet is rectangular. In FIG. 5 the side sheet is pentagon and in FIG. 6 the side sheet is trapezoid.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. An inflatable bioreactor bag for cell cultivation comprised of a top and a bottom sheet of polymer material that are joined along their edges to form a sealed bag, wherein two opposing edges are formed as clamping edges to allow clamping of said bioreactor bag to a rocker type bioreactor, wherein the top sheet is at least 5% longer than the bottom sheet between the clamping edges.

2. The bioreactor bag of claim 1, wherein the bioreactor bag comprises intermediate side sheets interconnecting the top and a bottom sheet.

3. The bioreactor bag of claim 1, wherein the top sheet is enlarged on two sides.

4. The bioreactor bag of claim 1, wherein the bottom sheet is enlarged on two sides.

5. The bioreactor bag of claim 1, wherein the side sheet has one edge of the equal length as the bottom sheet.

6. The bioreactor bag of claim 1, wherein the length between the non-clamping edges of the top sheet are formed to be of equal length as the bottom sheet between the non-clamping edges.

7. The bioreactor bag of claim 1, wherein the side sheets are curved.

8. The bioreactor bag of claim 1, wherein the side sheets are triangular.

9. The bioreactor bag of claim 1, wherein the side sheets are rectangular.

10. The bioreactor bag of claim 1, wherein the side sheets are pentagon.

11. The bioreactor bag of claim 1, wherein the side sheets are trapezoid.
